(19) United States
(12) Patent Application Publication
(10) Pub. No.: US 2003/0095937 A1
(43) Pub. Date: May 22, 2003

(54) Method for stimulating hair growth by administering vitamin D analogs

(76) Inventors: H. Philip Koeffler, Los Angeles, CA (US); Vijay Vegesna, Los Angeles, CA (US)

Correspondence Address: PILLSBURY WINTHROP LLP, Suite 2800, 725 South Figueroa Street, Los Angeles, CA, 90017-5406, US

(21) Appl. No.: 09/969,380

(22) Filed: Oct. 2, 2001

(51) Int. Cl. A61K 31/59; A61K 7/06

(52) U.S. Cl. 424/70.1; 514/167

(57) ABSTRACT

Disclosed herein is a method for stimulating the growth of hair by administering to a subject analogs of vitamin D₃ or prodrugs of these analogs. Disclosed are doses and routes for such analogs, as well as experimental data that show the analogs of the invention are effective in stimulating hair growth even in nude mice (BNX nu/nu mice).

### FIELD OF THE INVENTION

Embodiments of the present invention are directed to a method for promoting the growth of hair in mammals.

### BACKGROUND OF THE INVENTION

Men have pursued a cure for baldness as King Arthur's knights pursued the Holy Grail. We need not dwell on why this is so; hair has long been a symbol of masculinity and a tool by which women express their femininity. Many people have attempted, and failed, to provide an effective treatment. Perhaps it was really the disappointment this failure caused that prompted Cicero to say, “It is foolish to tear one's hair in grief, as though sorrow would be made less with baldness.”

Those seeking a cure for hair loss have applied to their scalp compounds such as ascorbic acid, benzoic acid, retinoic acid, estradiol, wheat germ oil, earth metals, and sulfur. One inventor, DiTucci (U.S. Pat. No. 5,674,510) for example, teaches that a mixture of garlic powder, brewer's yeast, grapefruit juice, acetic acid, and kelp can “eliminate” hair loss. Despite such efforts, however, a basic medical text still teaches that “[t]herapeutic options for male-pattern alopecia are limited.” M. H. Beers and R. Berkow, eds., Merck Manual of Diagnosis and Therapy, §10, Ch. 116 (Centennial Edition, 1999).

The U.S. Food and Drug Administration has yet to approve the sale of any non-prescription drug that claims it prevents hair loss or promotes the growth of new hair. The FDA has gone to court to stop the sale of such drugs, arguing (and always winning) that there is no scientific evidence that supports these claims. The FDA has approved only two drugs for treating hair loss, minoxidil and finasteride, and these drugs have only limited effectiveness. Only about half of men and women respond positively to these drugs; of those individuals who report a positive responsive, the response tends to be a modest one, with most individuals reporting only moderate hair growth. These drugs are moreover expensive, require several months (typically 6 to 9 months or more) to produce any observable effect, and exclude large categories of potential users (minoxidil is recommended only for use in healthy individuals without any scalp abrasions; finasteride is contraindicated in women who are or may be pregnant because it may cause abnormalities of the external genitalia of the male fetus).

Ever since Cicero's time and probably even before it, there has been a significant need for an effective method to promote the growth of hair and prevent its loss. Disclosed herein is such a method.

### SUMMARY OF THE INVENTION

It is an object of the invention to provide a method of promoting the growth of hair and preventing its loss, to provide compositions for achieving this object, and to address the deficiencies of the prior art, namely, the production of only modest growth in only some individuals, only after many months of expensive treatment.

Disclosed herein is a method of promoting the growth of hair and preventing its loss by administering to a subject analogs of vitamin D₃. These analogs comprise compounds of Formula I:

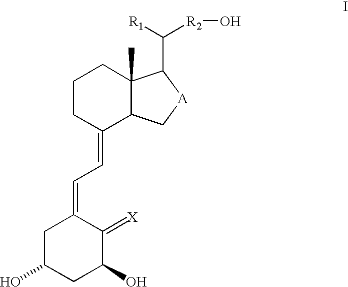

or pharmaceutically acceptable salts thereof, wherein R¹ and R² are each independently selected from the group consisting of

a) a saturated or unsaturated straight hydrocarbon having 3-15 C atoms;

b) a saturated or unsaturated straight hydrocarbon having 3-15 C atoms, wherein at least one C atom is substituted with a substituent independently selected from the group consisting of OH, F, alkyl (C₁₋₃), alkenyl (C₁₋₃) and cycloalkyl (C₃₋₅);

c) a saturated or unsaturated straight oxahydrocarbon group having 3-15 C atoms;

d) a saturated or unsaturated straight oxahydrocarbon group having 3-15 C atoms, wherein at least one C atom is substituted with a substituent independently selected from the group consisting of OH, F, alkyl (C₁₋₃), alkenyl (C₁₋₃) and cycloalkyl (C₃₋₅);

e) C₃₋₆-cycloalkyl;

f) an aromatic group; and

g) an aromatic group substituted with at least one halogen, C₁₋₃-alkyl, or alkoxy;

wherein X is methyl, methylene, or is absent; and

wherein A is a double or single bond.

In a preferred embodiment, one administers analogs of Formula II:

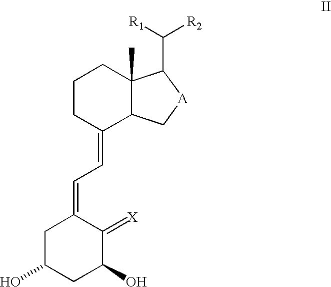

or pharmaceutically acceptable salts thereof, wherein R¹, R², and Z are selected from the group consisting of

R¹=H, R²=CH—CH—CH—C(CH₂CH₃)₂—OH, X is methylene, and A is a single bond;

b) R¹=CH₂—CH₂—CH₂—C(CH₃)₂—OH, R²=CH₂—CH₂—CH₂—C(CH₃)₂—OH, X is absent, and A is a single bond;

c) R¹=cyclopropyl, R²=CH₂—CH—CH—C(CH₃)—OH, X is absent, and A is a single bond;

d) R¹=H, R²=CH₂—CH₂—CO—C(CH₃)₂—OH, X is absent, and A is a double bond;

e) R¹=H, R²=CH₂—CH—CH—C(CF₃)—OH, X is absent, and A is a double bond; and

f) R¹=H, R²=O—CH₂—CH₂—CH₂—C(CH₂CH₃)₂—OH, X is methylene, and A is a single bond.

The compound as defined in a) is an analog of vitamin D₃ known commercially as EB1089; the compound as defined in b) is an analog of vitamin D₃ known as Ro27-5646; the compound as defined in c) is an analog of vitamin D₃ known as Ro27-0574; the compound as defined in d) is an analog of vitamin D₃ known as Ro26-9114; and the compound as defined in e) is 1,25-(OH)₂-16-ene-23-yne-26,27-F6-19-nor-D₃, an analog of vitamin D₃ known as Ro25-9022; the compound as defined in f) is 1α, 25-(OH)₂20-epi-22-oxa-24,26,27-trishomo-vitamin D₃, an analog of vitamin D₃ known as KH1060.

Administering compounds of the invention to a subject produces significant hair growth, even in subjects with congenital alopecia. In the experiments described herein, the inventors show that compounds of the invention can stimulate hair growth even in nude mice, that is, mice with a genetic mutation that completely prevents them from growing healthy hair. Compounds of the invention produce far less side effects (hypercalcemia) than vitamin D₃ and even its physiologically active form (1,25(OH)₂D₃), which is 100 times more potent. These compounds may be administered topically, orally, or parenterally, but are preferably administered orally.

### BRIEF DESCRIPTION OF THE FIGURES

FIG. 1 is a photograph comparing two nude mice, one of which was treated according to the method of invention, the other of which was not. The upper mouse received a diluant; the lower mouse received a vitamin D₃ analog (Ro25-9022) at 0.0625 μg intraperitoneally 3 times per week for 36 days. Both mice were sacrificed at the end of 36 days to examine their hair histology.

FIG. 2A shows skin from a control nude mouse with abortive hair follicles and rudimentary pilosebaceous units. FIG. 2B shows skin from a test mouse receiving the treatment shown in (and described in the caption of) FIG. 1.

FIG. 3 shows the effect of vitamin D₃ analogs on hair growth in nude mice. FIG. 3A compares mice treated with Ro27-0574 (--□--) and mice treated with a control (-♦-); FIG. 3B compares Ro25-9022 (--□--) and control (-♦-); FIG. 3C compares Ro26-9114 (--□--) and control (-♦-); FIG. 3D compares Ro27-5646 (--□--) and control (-♦-); FIG. 3E compares KH1060 (--□--) and control (-♦-); and FIG. 3F compares male nude mice treated with EB1089 (--□--), female nude mice treated with EB1089 (--Δ--), and control (-♦-). Mice were scored 3 times per week.

FIG. 4 shows the effect of vitamin D₃ analogs on expression of keratins in the skin of nude mice. FIG. 4A compares mice treated with Ro27-0574 (--□--) and mice treated with a control (-♦-); FIG. 4B compares Ro25-9022 (--□--) and control (-♦-); FIG. 4C compares Ro26-9114 (--□--) and control (-♦-); FIG. 4D compares Ro27-5646 (--□--) and control (-♦-); FIG. 4E compares KH1060 (--□--) and control (-♦-); and FIG. 4F compares male nude mice treated with EB1089 (--□--), female nude mice treated with EB1089 (--Δ--), and control (-♦-).

### DETAILED DESCRIPTION OF THE INVENTION

The hair follicle does not grow continuously throughout its life, but passes through three stages which together comprise the pilar cycle. The anagen stage is the growth stage, and normally lasts three to seven years in humans. During the catagen stage, growth stops and hair follicles atrophy. It lasts about three to four weeks. The telogen stage is the resting stage, during which the hair follicle progressively separate and finally fall out. It lasts about three to four months. Normally 80 to 95 percent of the follicles are in the anagen phase, less than 1 percent are in the catagen phase, and the rest are in the telogen phase.

Alopecia results when the pilar cycle is disturbed, resulting in excessive hair loss. In the typical case, the anagen phase terminates prematurely; hair follicles stop growing, the catagen phase appears early, and the follicles, often a large number of them, proceed to the telogen phase, where they fall out. The molecular signals that control the transition of the follicles between these stages is not understood.

### Vitamin D₃ Analogs of the Invention

The method of the present invention promotes the restoration of a normal pilar cycle. In its most significant aspect, the method stimulates the growth of hair, thereby prolonging the anagen stage; it can do so even in experimental animals that have been genetically engineered not to produce any healthy hair at all.

Compounds of the invention are analogs of 1,25(OH)₂-D₃, the physiologically active form of vitamin D₃. “Analog” has here its standard meaning, “a chemical compound that is structurally similar to another but differs slightly in composition,” Merriam Webster's Medical Desk Dictionary (1997), and any analog of vitamin D₃ may be used. Accordingly, in one embodiment of the invention, one stimulates the growth of hair in a subject by administering to the subject a compound having the following formula:

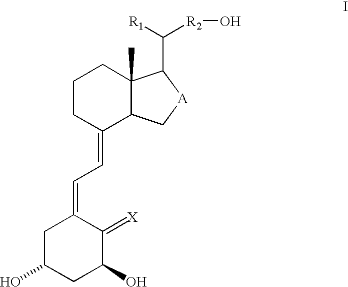

or pharmaceutically acceptable salts thereof, wherein R¹ and R² are each independently selected from the group consisting of

a) a saturated or unsaturated straight hydrocarbon having 3-15 C atoms;

b) a saturated or unsaturated straight hydrocarbon having 3-15 C atoms, wherein at least one C atom is substituted with a substituent independently selected from the group consisting of OH, F, alkyl (C₁₋₃), alkenyl (C₁₋₃) and cycloalkyl (C₃₋₅);

c) a saturated or unsaturated straight oxahydrocarbon group having 3-15 C atoms;

d) a saturated or unsaturated straight oxahydrocarbon group having 3-15 C atoms, wherein at least one C atom is substituted with a substituent independently selected from the group consisting of OH, F, alkyl (C₁₋₃), alkenyl (C₁₋₃) and cycloalkyl (C₃₋₅);

e) C₃₋₆-cycloalkyl;

f) an aromatic group; and

g) an aromatic group substituted with at least one halogen, C₁₋₃-alkyl, or alkoxy;

wherein X is methyl, methylene, or is absent; and

wherein A is a double or single bond.

In a preferred embodiment, one stimulates the growth of hair in a subject by administering to the subject a compound having the following formula:

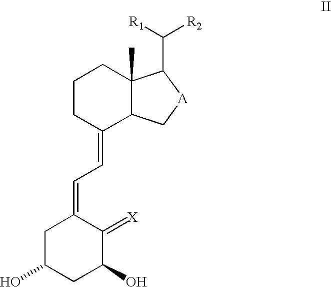

or pharmaceutically acceptable salts thereof, wherein R¹, R², and Z are selected from the group consisting of

a) R¹=H, R²=CH—CH—CH—C(CH₂CH₃)₂—OH, X is methylene, and A is a single bond;

b) R¹=CH₂—CH₂—CH₂—C(CH₃)₂—OH, R²=CH₂—CH₂—CH₂—C(CH₃)₂—OH, X is absent, and A is a single bond;

c) R¹=cyclopropyl, R²=CH₂—CH—CH—C(CH₃)—OH, X is absent, and A is a single bond;

d) R¹=H, R²=CH₂—CH₂—CO—C(CH₃)₂—OH, X is absent, and A is a double bond;

e) R¹=H, R²=CH₂—CH—CH—C(CF₃)—OH, X is absent, and A is a double bond; and

f) R¹=H, R²=O—CH₂—CH₂—CH₂—C(CH₂CH₃)₂—OH, X is methylene, and A is a single bond.

The compound as defined in a) is an analog of vitamin D₃ known commercially as EB1089. It differs from 1,25(OH)₂—D₃in that it has an altered side chain comprising 26,27 dimethyl groups, an insertion of an extra carbon at C-24 (24a), and two double bonds at carbons 22,23 and 24,24a. It is widely available commercially, as are all of the analogs of the invention; one commercial source is Leo pharmaceutical Products of Denmark. The compound has the following formula:

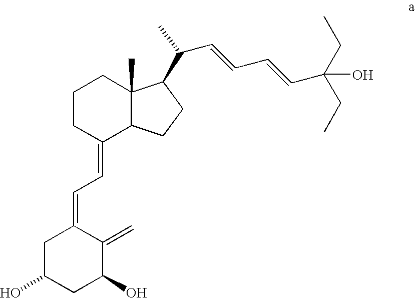

The compound as defined in b) is an analog of vitamin D₃ known as Ro27-5646. It has two side-chains and no C-19 alkyl group. It is available from Hoffmann LaRoche, Inc., of Nutley, N.J. (“Hoffman LaRoche”). It has the following formula:

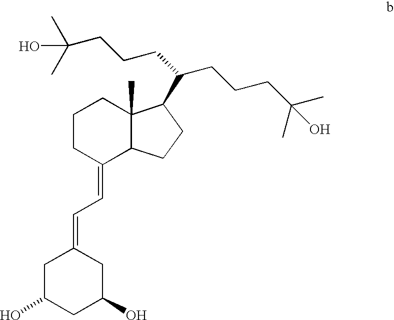

The compound as defined in c) is an analog of vitamin D₃ known as Ro27-0574. It has desaturation of the side-chain, 6 fluorines added to the side-chain, and propylene added to the C-17. It is available from Hoffmann LaRoche. It has the following formula:

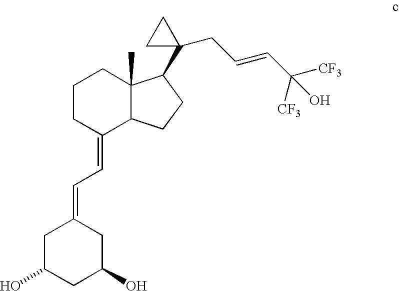

The compound as defined in d) is an analog of vitamin D₃ known as Ro26-9114. It has a double bond at C-16 and an oxygen molecule attached to the side-chain. It is available from Hoffmann LaRoche. It has the following formula:

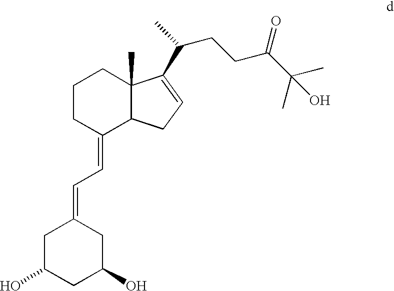

The compound as defined in e) is 1,25-(OH)₂-16-ene-23-yne-26,27-F6-19-nor-D₃, an analog of vitamin D₃ known as Ro25-9022. It has 6 fluorines on the side chain, desaturation of the side-arm, and removal of the C-19 alkene. It is available from Hoffmann LaRoche. It has the following formula: y

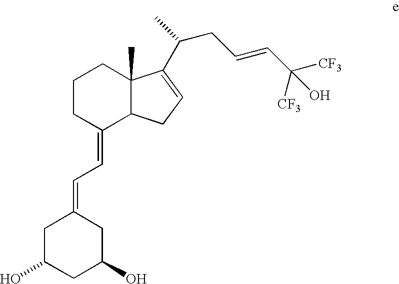

The compound as defined in f) is 1α, 25—(OH)₂-20-epi-22-oxa-24,26,27-trishomo-vitamin D₃, an analog of vitamin D₃ known as KH1060. It has an extension of the side-arm and an addition of an oxygen to the side-arm. It is available from Leo Pharmaceutical Products.

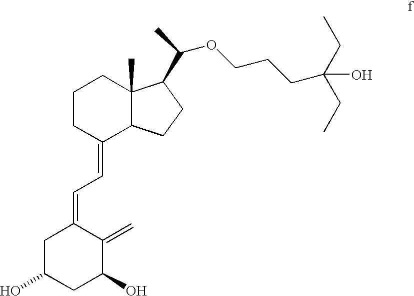

Any of the analogs of the invention may be delivered as pharmaceutically acceptable salts. As used herein, the term “pharmaceutically acceptable salt” refers to any salt which is non-toxic to living organisms. Typical pharmaceutically acceptable salts include those salts prepared by reaction of the compounds of the present invention with a mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid and the like, and organic acids such as p-toluenesulfonic, methanesulfonic acid, oxalic acid, p-bromophenylsulfonic acid, carbonic acid, succinic acid, citric acid, benzoic acid, and acetic acid.

Examples of pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, thydroxybutyrate, glycollate, tartrate, methanesulfonate, propanesulfonate, naphthalene-1-sulfonate, napththalene-2-sulfonate, and mandelate salts.

Base addition salts include those derived from inorganic bases, such as ammonium or alkali or alkaline earth metal hydroxides, carbonates, bicarbonates, and the like. Some bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, and calcium carbonate.

The particular ion forming a part of any salt of this invention is not critical; what is critical is that the salt as a whole is pharmacologically acceptable and the ion does not contribute undesired qualities to the compound as a whole. Appropriate salts will be readily apparent to those of ordinary skill in the art.

In another embodiment of the invention, one stimulates the growth of hair in a subject by administering to the subject a prodrug of the compound of Formula I, or, preferably, a prodrug of the compound of Formula II.

As used herein, the term “prodrug” refers to any compound that is converted into an active analog of vitamin D₃ by metabolic processes within the body. There are several reasons why one might wish to administer a prodrug of a vitamin D₃ analog of the invention rather than the analog itself. Depending on the particular analog and/or the particular analog salt used, a prodrug might have superior characteristics as far as solubility, absorption, stability, release, toxicity, and patient acceptability are concerned. It should be readily apparent to one of ordinary skill in the art how one can make a prodrug of any analog of the invention. There are many strategies for doing so. One can replace one or more of the hydroxy groups in the side chain with hydrogen, for example. Such prodrugs are converted in vivo by enzymatic hydroxylation to active vitamin D₃ analogs. Other prodrugs should be readily apparent to one of ordinary skill in the art.

### Route of Administration

The compounds described herein can be administered to a subject via several routes, including, for example, parenterally, orally, topically, and intraperitoneally. Administration via the oral route is preferred.

Routes of parenteral administration include, for example, intravenous, intramuscular, interstitial, intra-arterial, subcutaneous, intrasynovial, and transepithelial (including transdermal) injection. For both parenteral or intraperitoneal administration, compounds of the invention are prepared as a solution as either the free base or a pharmaceutically acceptable salt. Any solvent in which vitamin D compounds are administered may be used to administer the compounds of the invention; such solvents are widely known in the art. A buffered saline solution is preferred.

Pharmaceutical forms suitable for injectable use include sterile aqueous solutions, dispersions, and sterile powders from which injectable solutions or dispersions may be prepared. In all cases, the form should be sterile, stable under the conditions of manufacture and storage, and should be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size (in the case of a dispersion) and by the use of surfactants. Preventing microbial action can be brought about by various antibacterial and antifungal agents, for example, parabens, chlorobutanol, phenol, sorbic acid, thimerosal, and the like. In many cases, it will be preferable to include agents, such as sugars or sodium chloride, that allow one to achieve a desired tonicity.

Sterile injectable solutions are prepared by incorporating a compound of the invention in the appropriate solvent with various of the other ingredients enumerated above, followed by filtered sterilization. Dispersions are generally prepared by incorporating a compound of the invention into a sterile vehicle which contains the basic dispersion medium and any of the other ingredients enumerated above. Sterile powders are prepared by vacuum drying and/or freeze drying mixtures that form injectable solutions or dispersions when solvent is added.

Formulations suitable for oral administration are prepared by uniformly combining a compound of the invention with a pharmaceutically acceptable liquid carrier, a finely divided solid carrier, or both, and then shaping the product if necessary. As used herein, “pharmaceutically acceptable carrier” refers to a carrier that is compatible with the compounds of the invention and does not harm the subjects to which it is administered. Suitable pharmaceutically acceptable carriers include, for example, water, alcohols, natural or hardened oils and waxes, calcium and sodium carbonates, calcium phosphate, kaolin, talc, and lactose.

An oral formulation may contain one or more excipients, including the following: preservatives, such as ethyl-p-hydroxybenzoate; suspending agents such as methyl cellulose, tragacanth, and sodium alginate; wetting agents such as lecithin, polyoxyethylene stearate, and polyoxyethylene sorbitan mono-oleate; granulating and disintegrating agents such as starch and alginic acid; binding agents such as starch, gelatin, and acacia; lubricating agents such as magnesium stearate, stearic acid, and talc; and flavoring and coloring agents.

Oral formulations may be presented in any of the following forms: discrete units such as capsules, cachets, or tablets each containing a predetermined amount of the active ingredient; powder or granules; solutions or suspensions in an aqueous liquid or a non-aqueous liquid; as oil-in-water liquid emulsions or water-in-oil emulsions; as lozenges, pastilles, mouthwashes, and any other form known in the art that is suitable for oral administration.

Formulations suitable for topical administration may be presented as creams, ointments, shampoos, and lotions. Suitable carriers for such formulations include vegetable or mineral oils, white petrolatum (white soft parrafin), branched chain fats or oils, animal fats and high molecular weight alcohol (greater than C₁₂). Preferred carriers are those in which the compounds of the invention are soluble. Emulsifiers, stabilizers, humectants and antioxidants may also be included as well as agents imparting color or fragrance, if desired.

### Doses for Oral or Parenteral Administration

Compounds of the invention are administered in doses effective to produce the growth of hair. Where the subject to be treated is a human, effective amounts of the compounds of the invention generally range from 0.1 μg to 25 μg of vitamin D₃ analog (or salt or prodrug of the analog) per day. The principal side effect of oral and parenteral administration is hypercalcemia. Although analogs of 1,25(OH)₂D₃ have a lower tendency to produce hypercalcemia than 1,25(OH)₂D₃ does, hypercalcemia may result in some individuals when the analogs are administered in amounts greater than 25 μg per day.

The presently preferred dose is approximately 0.25 μg to 1 μg per day. One of skill in the art can readily determine the dosage of the compounds of the invention which will be most suitable for prophylaxis or treatment, and would know that the dosage may vary with the form of administration and the particular compound chosen, and also, that the dosage may vary with the particular patient.

### Possible Mechanism of Action

A variety of hormones have effects on hair growth including estrogen, growth hormone, and androgens. Androgens can promote miniaturization of follicles and shortening of the duration of the anagen stage. In contrast, estrogen can prolong the anagen stage. Minoxidil prolongs the anagen stage of hair growth and causes follicles at rest to enlarge and grow. It lengthens and enlarges the small vellus hairs and decreases shedding, but how it mediates these effects is unclear. Finasteride is a 5α-RA type 2 inhibitor and thus decreases serum and cutaneous dihydrotestosterone levels and therefore inhibits androgen-dependent miniaturization of hair follicles.

Hypertrichosis or hirsuitism with growth of hair over all of the body is a side-effect frequently observed in transplant patients who receive the immunosuppressant cyclosporin A (CsA) or FK506. Although these drugs differ from each other in structure, both inhibit T-cell activation by interfering with IL-2 production. The mechanism of inhibition of IL-2 gene expression may be through inhibition of calcineurin by Ca²⁺/calmodulin-dependent phosphatase pathway. This might also influence the calcium concentration at the hair follicular level with an indirect effect on local androgen levels. Nude mice treated with CsA can also grow hair and this can not be the result of interfering with T-cell activation because nude mice do not have T-cells. However, the skin of these mice have an increased level of 5α-dihydroxytestosterone (5α-DHT) as a result of an increased activity of 5α-reductase (5α-RA)(14). But how this might stimulate hair growth is unclear.

Ligands of nuclear hormone receptors can have effects on hair growth. Thyroxine, for example, can at either high or low levels cause transient shedding of hair. Also, ligands of retinoic acid receptor (RAR) can cause premature onset of the catagen stage with premature loss of hair

Compounds of the invention are analogs of vitamin D₃. The physiologically active form of this vitamin, 1,25(OH)₂-D₃, and its analogs such as calcipotriol (MC903) and tacalcitol are used successfully in topical treatment of psoriasis as well as scleroderma. These drugs cause a decrease in keratinocyte proliferation and increases their differentiation. Keratinocytes can convert inactive androgens such as dehydroepiandrosterone to an active androgen (dihydrotestosterone) by way of 5α-RA. Likewise, the hair follicle can express several steroidogenic enzyme with 17β-ydroxysteroid dehydrogenase (17β-HSD). The 17βHSD changes estradiol to inactive estrone. 1,25(OH)₂-D₃ can increase the activity of 17βHSD in keratinocytes in the skin, and therefore convert local estradiol to estrone. Many skin cells express estrogen receptors; therefore, the 17β-HSD stimulatory activity may provide an antiproliferative effect by decreasing the mitogenic effects of estradiol. Nevertheless, how these effects might enhance hair growth is unclear.

Hair follicle growth depends on the WNT β-catenin stimulation of TCF4 transcription factor which stimulates expression of a number of other growth related products. Whn (Foxn1) and bone morphogenic protein (BMP) may stimulate this pathway. For example, when β-catenin is conditionally ablated, BMP expression is impaired and growth at hair follicles is blocked. The phenotype of these mice is very similar to HR or RXRα conditional knockout mice suggesting that these pathways may be interconnected through the WNT signaling pathway. In fact, expression of Whn, hoxc13, Msx1 and Msx2 are strongly reduced or absent when the WNT β-catenin pathway is disrupted. This suggests that control of Whn expression may be downstream of TCF4 transcription.

The compounds of the invention have been previously shown to have greater abilities to induce differentiation and inhibit proliferation of a variety of cancer related cell types including leukemia and breast cancer cells. Furthermore, these analogs produce less hypercalcemia than 1,25(OH)₂D₃ does. The mechanism by which these analogs have this enhanced anti-proliferative, prodifferentiation effects are unclear. Each of the analogs is able to bind to VDR. Some of the mechanisms that are hypothesized for their increase potency include the fact that they have decrease binding to D binding protein in the serum, which allows more ready access to entering the cell. Some data suggest that these analogs alter the conformation of the vitamin D receptor making it more active or enhances the half-life of the vitamin D receptor. The overall affinity of the analog for the receptor does not appear to be much different than 1,25(OH)₂D₃. It could also be altering the ability of co-repressors or co-activators to interact with the ligand activated receptor. Whatever the cause may be, these analogs are acting on the hair follicle probably by stimulating the expression of genes that are downstream of the Whn transcription factor and turning these genes on in order to stimulate hair growth.

### EXAMPLES

The following examples are typical of the procedures that may be used to stimulate hair growth according to the methods of the invention. Modifications of these examples will be apparent to those skilled in the art who seek to stimulate hair growth in different subjects or with different doses than the ones administered here.

### Subjects

A gene known as hairless (HR), which is a single zinc finger transcription factor, heterodimerizes with thyroid receptor. Mice with germlne mutation of HR lose all of their hair. One particular mutation of the HR gene, known as rhino allele of the HR gene, results in alopecia and abnormalities of the inner ear, retina and colon as well as severe wrinkling of the skin. These mice have numerous, large cysts.

RXRα conditional knockout mice have a normal first coat of hair, but then subsequently lose their hair. The RXRα probably interacts with another nuclear hormone receptor and the ligand to this receptor probably maintains the dermal papilla connection with the hair follicle.

Vitamin D receptor (VDR) knockout mice are characterized by a 1,25(OH)₂-D₃ resistant rickets with hypocalcemia, hypophosphatemia, hyperparathyroidism, rickets, osteomalacia, and alopecia. Placing these animals on a high calcium, phosphorous and lactose diet can normalize the phenotype except for the alopecia. Furthermore, individuals with vitamin D₃ resistant rickets type 2 also have a germlne mutation of VDR with either partial or total alopecia. The VDR knock-out mouse has alopecia which is a result of aberrant initiation of the anagen phase. A set of experiments using a hair reconstitution assay showed that normal morphogenesis of hair follicles would occur irrespective of the VDR status of the keratinocytes and dermal papilla cells, but the hair follicles reconstituted with VDR knock-out keratinocytes showed a defective response to initiation of the anagen cycle suggesting that epithelial-mesenchymal communication is needed for normal hair cycling. The studies suggested that keratinocytes were the aberrant cell in the hairless VDR knock-out mice.

Keratinocytes express Whn and have also been implicated as the defective cell type in the nude mouse. Recombinant keratinocyte growth factor was shown to induce dose-dependent hair growth by stimulating proliferation and normal differentiation of follicular keratinocytes. In hair reconstitution assays, recombining nude keratinocytes with wild-type follicular papilla cells in a skin graft result in development of nude follicles. One study demonstrated that expression of several keratin genes was reduced in nude skin, and that keratin gene transcription could be regulated by Whn. The experiments described herein show that treatment of nude mice with vitamin D₃ analogs up-regulates expression of keratins in the mice's skin as they were growing hair. These observations suggest that vitamin D₃ analogs acts to normalize keratinocytes of nude mice, thereby allowing production of hair.

In the experiments described herein, BNX nu/nu male and female mice at 8 weeks of age were used. In one series of experiments, a total of 3 males were allotted equally within each of the groups consisting of 6 different vitamin D₃ compounds, and a control untreated group. In an addition, three male and 3 female mice received EB1089. All of the analogs were injected intraperitoneally (i.p.), three times a week in a volume of 200 μl phosphate buffered saline.

### Preparation of Vitamin D₃ Analogs

Vitamin D₃ analogs were dissolved in absolute ethanol prior to determining the molar concentration as measured by UV absorbance using their molar extinction coefficient at 264 nmol/liter. All the analogs were stored at −20° C. in absolute ethanol as stock solutions of 10⁻³ mol/liter. The last dilution was made in phosphate buffered saline, prior to administration into mice. Vitamin D₃ compounds were always protected from light. The inventors chose the highest dose that would not cause hypercalcemia. These doses, identified in previous experiments, are summarized below in Table 1.

TABLE 1
Summary of calcemic properties of Vitamin D₃ analogs

| | ED50* | | | |
|---|---|---|---|---|
| | HL60 | MCF-7 | | |
| | MOLAR | MOLAR | NON-HYPERCALCEMIC | |
| COMPOUND | UNITS | UNITS | DOSE | |
| Ro 27-5646 | 2 × 10⁻¹⁰ | 4 × 10⁻¹⁰ | 0.01 | μg/mouse |
| Ro 27-0574 | 2 × 10⁻¹⁰ | 2 × 10⁻⁹ | Not done | |
| EB 1089 | 5 × 10⁻¹¹ | | 0.5 | μg/kg |
| Ro 26-9114 | 1 × 10⁻¹⁰ | | 6 | μg/mouse |
| Ro 25-9022 | 2 × 10⁻¹⁰ | 6 × 10⁻¹⁰ | 0.00125 | μg/mouse |
| KH 1060 | 1 × 10⁻¹² | 1 × 10⁻¹⁰ | 0.0125 | μg/mouse |
| 1,25(OH)₂D₃ | 1 × 10⁻⁸ | 1 × 10⁻⁷ | 0.0626 | μg/mouse |

*ED50 refers to the effective dose, in molar units, that inhibited 50% clonal growth of human HL60 myeloid leukemia cells and MCF-7 breast cancer cells.

### Scale for Monitoring Hair Cycle in BNX nu/nu Mice

The inventors developed the following scale for measuring hair growth in BNX mice:

TABLE 2
Scale for measuring hair growth

| SCORE | DESCRIPTION |
|---|---|
| 1 | Skin pink, no hair |
| 2 | Skin thick, pigmented, no hair |
| 3 | Skin thick, highly pigmented, no hair |
| 4 | Skin thick, pigmented, scattered hair |
| 5 | Hair 1-10% of area |
| 6 | Hair 10-25% of area |
| 7 | Hair 25-50% of area |
| 8 | Hair 50-75% of area |
| 9 | Hair >75% of area |
| 10 | Hair 100% covered |

### Histological Analysis Hair Follicles

Skin samples were obtained after treatment with either vitamin D₃ compounds or control diluant. They were fixed for histological analysis, stained with hematoxylin/eosin and examined by microscopy.

### RNA Analysis

Skin samples from mice were processed as is routinely done in the art. RT-PCR was performed to make cDNA which was examined for expression of CYP19 gene that encodes the P450 aromatase (9). This enzyme has been found to be elevated after with exposure of cells to 1,25(OH)₂D₃ and is required for changing androstendione to testosterone (8). The P450 aromatase is localized to the external root sheath during anagen, but not telogen, indicating that it plays a role in the hair cycle by regulating the level of androgens formed locally.

### Results

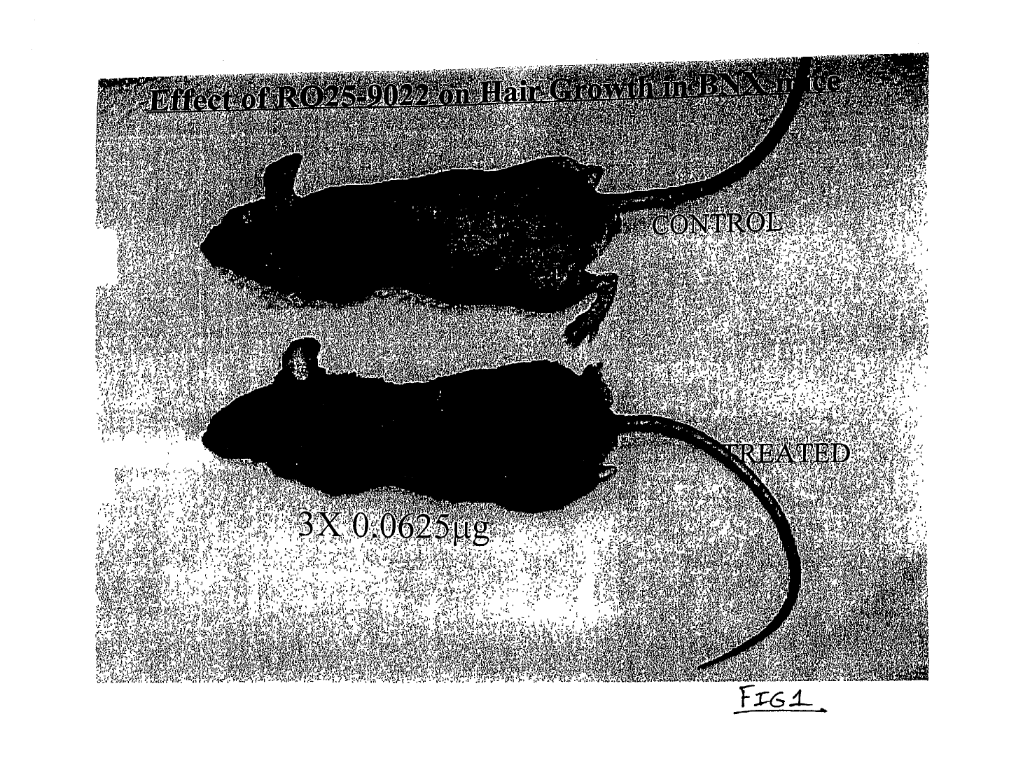

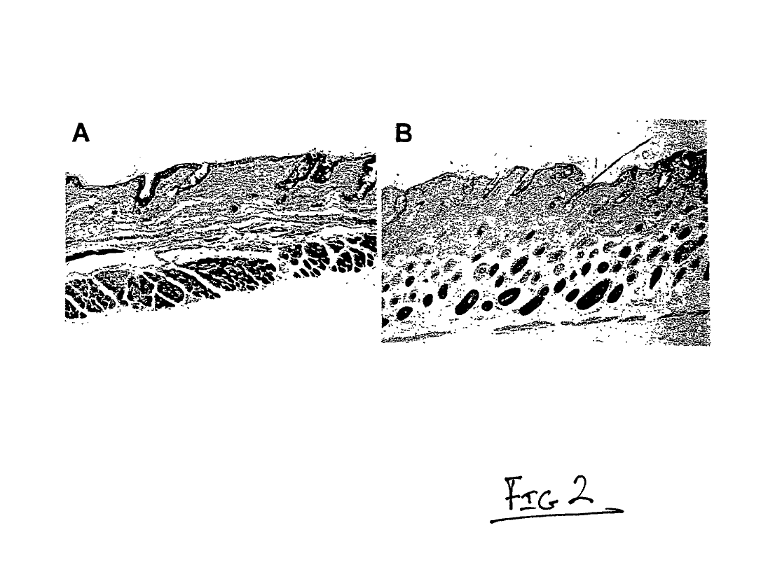

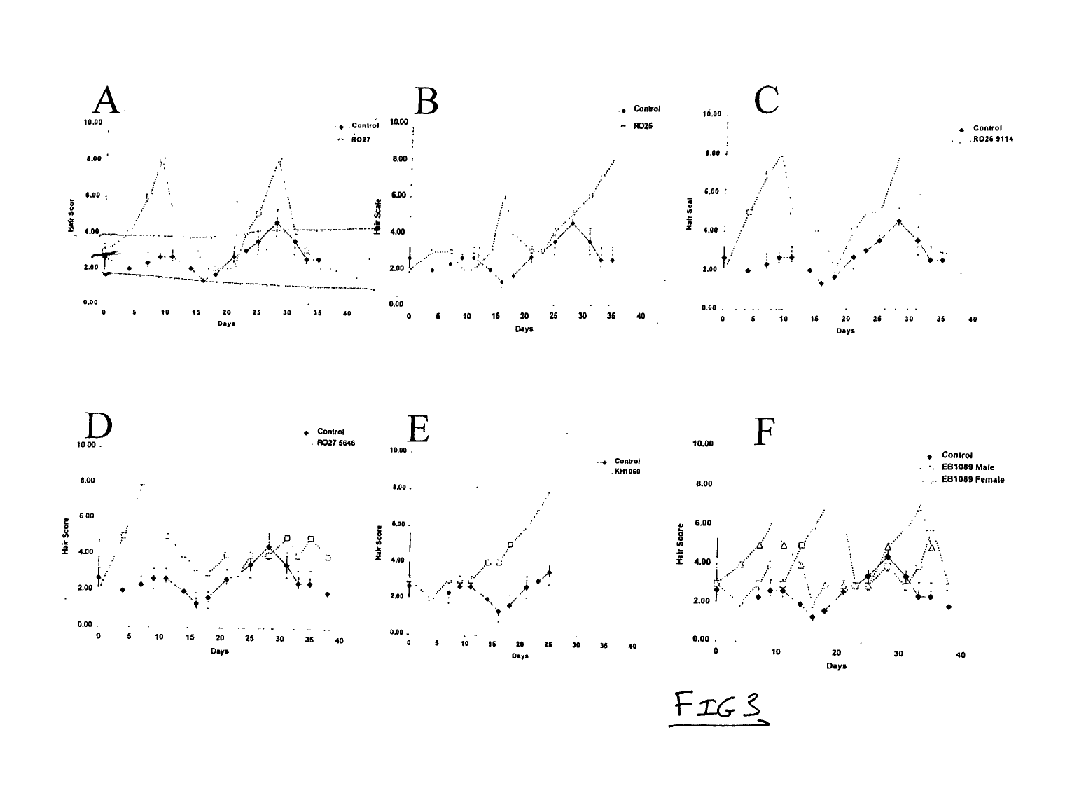

Nude (BNX nu/nu) mice received vitamin D₃ compounds as described herein or diluant control at a dose not associated with hypercalcemia, the major toxicity associated with these compounds. These mice developed hair as shown in FIGS. 1 and 2. Morphological examination confirmed that the vitamin D₃ analogs stimulated hair follicles to form (FIG. 2) Microscopic observation of skin samples at a peak score of 8 from the vitamin D₃ treatment groups showed a fully formed hair follicle; in contrast, no hair follicle or only a distorted one was found in the control samples. Histological slides were also stained with specific antibodies for keratins that are required for hair growth. All samples including the controls stained for keratins (data not shown). The appearance of hair after initiation of vitamin D injection varied somewhat from mouse to mouse and between the vitamin D analogs (FIG. 3).

The inventors examined hair growth of nude mice as the mice received diluant (control mice), 1.25(OH)₂D₃ or one of 6 vitamin D₃ analogs. Each group contained three mice. The effect of Vitamin D₃ analogs on hair growth of nude mice was systematically assessed on each mouse by giving them scores from a scale of 1-10 (Table1), 3 times a week. The score for hair growth takes in to account the thickness and pigmentation, as both of these characteristics correlate indirectly with induction of anagen, the growth phase of the hair cycle. For example, a score of 5 represents skin thickening with heavy pigmentation; this reflects the formation of the hair shaft prior to the protrusion of hair above the skin surface. Occasionally, diluant controls and those receiving 1,25(OH)₂D₃ reached this score, but did not exceed it. mmm

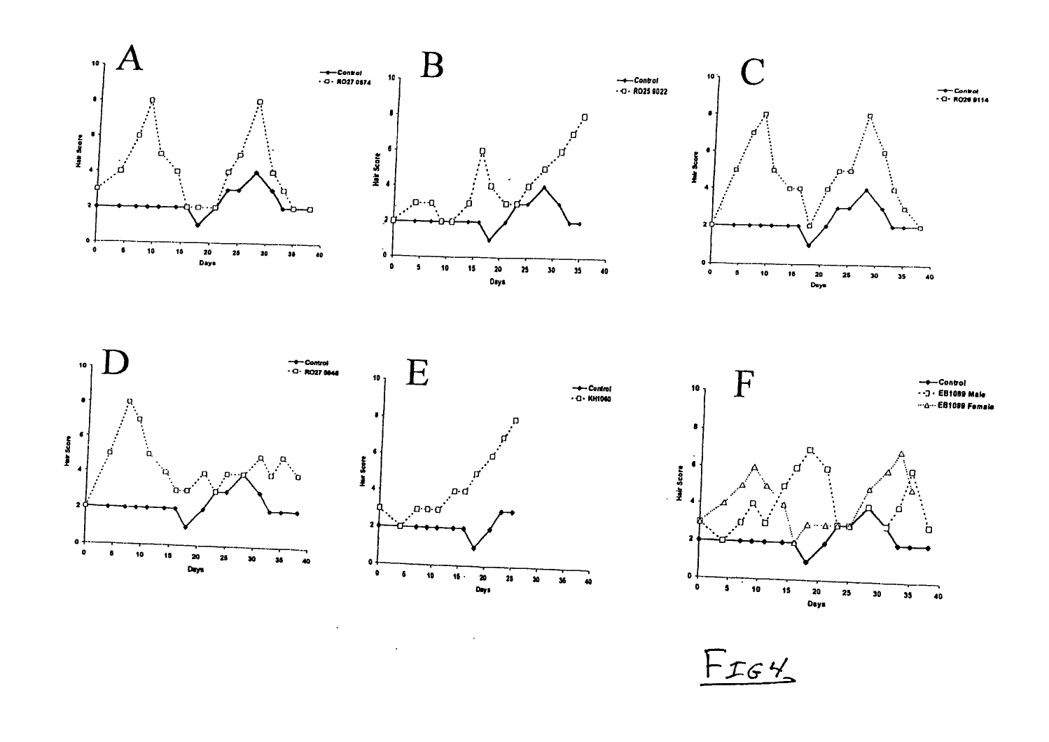

In the experimental groups, 13 of 30 male mice reached the peak score of either 7 or 8 for each of the 6 Vitamin D₃ analogs (FIGS. 3 and 4). The maximum hair growth score was consistently higher in mice treated with vitamin D₃ analogs (Table 3):

TABLE 3
Peak Hair Growth of Nude Mice Receiving Vitamin D₃ Compounds.

| | | | KH | Ro25- | Ro25- | Ro27- | Ro27- | EB1089 | EB1089 |
|---|---|---|---|---|---|---|---|---|---|
| | Control | 1,25(OH)₂D₃ | 1060 | 9022 | 9114 | 05 | 5646 | Males | Females |
| Peak | 5 | 5 | 8 | 8 | 8 | 8 | 8 | 7 | 7 |
| Score, | | | | | | | | | |
| Mouse 1 | | | | | | | | | |
| Peak | 4 | 5 | 6 | 4 | 8 | 8 | 8 | 8 | 7 |
| Score, | | | | | | | | | |
| Mouse 2 | | | | | | | | | |
| Peak | 3 | 4 | 6 | 4 | 4 | 4 | 6 | 4 | 7 |
| Score, | | | | | | | | | |
| Mouse 3 | | | | | | | | | |
| Mean | 4.00 | 4.67 | 6.67 | 6.33 | 5.33 | 6.67 | 7.33 | 6.33 | 7.00 |
| Peak | | | | | | | | | |
| Score | | | | | | | | | |
| Stand. | 1.00 | 0.58 | 1.15 | 2.08 | 2.31 | 2.31 | 1.15 | 2.08 | 0.00 |
| Deviation | | | | | | | | | |
| p Value | | 0.18 | 0.02 | 0.07 | 0.027 | 0.09 | 0.01 | 0.12 | 0.04 |

The nude mice received introperitoneal injections of a vitamin D₃ compound or diluant control 3 times a week. Their hair score was assessed at the same time using the scoring system shown in Table 1. The peak hair growth score for each mouse over at least 35 days of treatment is shown for both KH1060 and Ro25-9022. One mouse was sacrificed early (days 24 and 35, respectively) for hair morphology.

The compounds KH1060 (FIG. 3E), Ro27-5646 (FIG. 3D), and EB1089 (FIG. 3F) were the most consistently effective at inducing hair growth, as the mean peak score was significantly higher (p<0.05) than controls. A second cycle of hair growth (score 7-8) occurred in the Ro27-0574 (FIG. 3A), Ro269114 (FIG. 3C), and EB1089 (FIG. 3F) groups about 16 days from the first cycle. The speed to achieve maximal hair growth varied among the vitamin D₃ treatment groups. For example, the latency period was about 9 days for Ro25-9022, Ro26-9114 and EB1089 (female) groups. Latency was about 18 days for males receiving EB1089, 21 days for KH1060, and 36 days for those Ro25-9022.

The inventors also examined the ability of EB1089 to stimulate hair growth in male and female mice. Both males and females had hair stimulation but the first peak of hair growth in females slightly preceded the peak in the males (FIG. 3F).

Interestingly, hair lasted for about 6-9 days even though the vitamin D₃ analog treatment was discontinued. Skin samples at the highest score of 7 from Ro-25-9022 and control skin sample were processed to extract RNA, cDNA was made and amplified for the expression of CYP19 gene that encodes P450 aromatase. The experimental and control samples had equivalent expression of the aromatase (data not shown).

Those skilled in the art will recognize, or be able to determine using no more than routine experimentation, variations on the foregoing examples that will permit them to effectively treat subjects other than mice with the compounds of the invention.

What is claimed is:

1. A method of stimulating the growth of hair, the method comprising administering to a mammal an effective amount of a compound having the formula 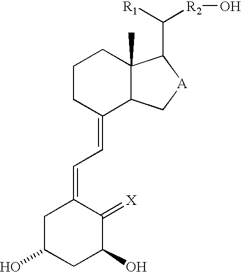
or pharmaceutically acceptable salts thereof, wherein R¹ and R² are each independently selected from the group consisting of
a) a saturated or unsaturated straight hydrocarbon having 3-15 C atoms;
b) a saturated or unsaturated straight hydrocarbon having 3-15 C atoms, wherein at least one C atom is substituted with a substituent independently selected from the group consisting of OH, F, alkyl (C₁₋₃), alkenyl (C₁₋₃) and cycloalkyl (C₃₋₅);
c) a saturated or unsaturated straight oxahydrocarbon group having 3-15 C atoms;
d) a saturated or unsaturated straight oxahydrocarbon group having 3-15 C atoms, wherein at least one C atom is substituted with a substituent independently selected from the group consisting of OH, F, alkyl (C₁₋₃), alkenyl (C₁₋₃) and cycloalkyl (C₃₋₅);
e) C₃₋₆cycloalkyl;
f) an aromatic group; and
g) an aromatic group substituted with at least one halogen, C₁₋₃-alkyl, or alkoxy;
X is methyl, methylene, or is absent; and
A is a double or single bond.

2. The method of claim 1, wherein the compound is administered via a route selected from the group consisting of oral, parenteral, and intraperitoneal administration.

3. The method of claim 1, wherein the mammal is a human.

4. The method of claim 3, wherein the compound is administered in an effective amount of between about 0.1 μg to about 25 μg mammal per day.

5. The method of claim 4, wherein the compound is administered in an effective amount of between about 0.25 μg to about 1 μg per day.

6. The method of claim 1, wherein the compound is administered topically.

7. A method of stimulating the growth of hair, the method comprising administering to a mammal an effective amount of a prodrug of a compound having the formula 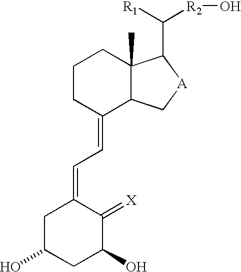
or pharmaceutically acceptable salts thereof, wherein R¹ and R² are each independently selected from the group consisting of
a) a saturated or unsaturated straight hydrocarbon having 3-15 C atoms;
b) a saturated or unsaturated straight hydrocarbon having 3-15 C atoms, wherein at least one C atom is substituted with a substituent independently selected from the group consisting of OH, F, alkyl (C₁₋₃), alkenyl (C₁₋₃) and cycloalkyl (C₃₋₅);
c) a saturated or unsaturated straight oxahydrocarbon group having 3-15 C atoms;
d) a saturated or unsaturated straight oxahydrocarbon group having 3-15 C atoms, wherein at least one C atom is substituted with a substituent independently selected from the group consisting of OH, F, alkyl (C₁₋₃), alkenyl (C₁₋₃) and cycloalkyl (C₃₋₅);
e) C₃₋₆-cycloalkyl;
f) an aromatic group; and
g) an aromatic group substituted with at least one halogen, C₁₋₃-alkyl, or alkoxy;
X is methyl, methylene, or is absent; and
A is a double or single bond.

8. The method of claim 7, wherein the compound is administered via a route selected from the group consisting of oral, parenteral, and intraperitoneal administration.

9. The method of claim 7, wherein the mammal is a human.

10. The method of claim 9, wherein the compound is administered in an effective amount of between about 0.1 μg to about 25 μg mammal per day.

11. The method of claim 10, wherein the compound is administered in an effective amount of between about 0.25 μg to about 1 μg per day.

12. The method of claim 7, wherein the compound is administered topically.

13. A method of stimulating the growth of hair, the method comprising administering to a mammal an effective amount of a compound having the formula 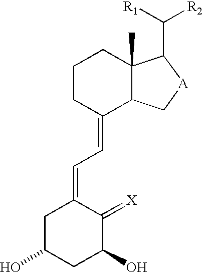
or pharmaceutically acceptable salts thereof, wherein R¹, R², and Z are selected from the group consisting of
a) R¹=H, R²=CH—CH—CH—C(CH₂CH₃)₂—OH, X is methylene, and A is a single bond;
b) R¹=CH₂—CH₂—CH₂—C(CH₃)₂—OH, R²=CH₂—CH₂—CH₂—C(CH₃)₂—OH, X is absent, and A is a single bond;
c) R¹=cyclopropyl, R²=CH₂—CH—CH—C(CH₃)—OH, X is absent, and A is a single bond;
d) R¹=H, R²=CH₂—CH₂—CO—C(CH₃)₂—OH, X is absent, and A is a double bond;
e) R¹=H, R²=CH₂—CH—CH—C(CF₃)—OH, X is absent, and A is a double bond; and
f) R¹=H, R²=O—CH₂—CH₂—CH₂—C(CH₂CH₃)₂—OH, X is methylene, and A is a single bond.

14. The method of claim 13, wherein R¹, X, and Z are as defined in a.

15. The method of claim 13, wherein R¹, X, and Z are as defined in b.

16. The method of claim 13, wherein R¹, X, and Z are as defined in c.

17. The method of claim 13, wherein R¹, X, and Z are as defined in d.

18. The method of claim 13, wherein R¹, X, and Z are as defined in e.

19. The method of claim 13, wherein R¹, X, and Z are as defined in f.

20. The method of claim 13, wherein the compound is administered via a route selected from the group consisting of oral, parenteral, and intraperitoneal administration.

21. The method of claim 13, wherein the mammal is a human.

22. The method of claim 21, wherein the compound is administered in an effective amount of between about 0.1 μg to about 25 μg mammal per day.

23. The method of claim 22, wherein the compound is administered in an effective amount of between about 0.25 μg to about 1 μg per day.

24. The method of claim 13, wherein the compound is administered topically.

25. A method of stimulating the growth of hair, the method comprising administering to a mammal an effective amount of a prodrug of a compound having the formula 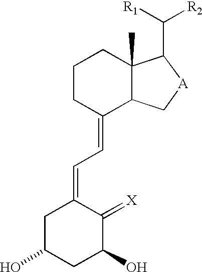
or pharmaceutically acceptable salts thereof, wherein R¹, R², and Z are selected from the group consisting of
a) R¹=H, R²=CH—CH—CH—C(CH₂CH₃)₂—OH, X is methylene, and A is a single bond;
b) R¹=CH₂—CH₂—CH₂—C(CH₃)₂—OH, R²=CH₂—CH₂—CH₂—C(CH₃)₂—OH, X is absent, and A is a single bond;
c) R¹=cyclopropyl, R²=CH₂—CH—CH—C(CH₃)—OH, X is absent, and A is a single bond;
d) R¹=H, R²=CH₂—CH₂—CO—C(CH₃)₂—OH, X is absent, and A is a double bond;
e) R¹=H, R²=CH₂—CH—CH—C(CF₃)—OH, X is absent, and A is a double bond; and
f) R¹=H, R²=O—CH₂—CH₂—CH₂—C(CH₂CH₃)₂—OH, X is methylene, and A is a single bond.

26. The method of claim 25, wherein R¹, X, and Z are as defined in a.

27. The method of claim 25, wherein R¹, X, and Z are as defined in b.

28. The method of claim 25, wherein R¹, X, and Z are as defined in c.

29. The method of claim 25, wherein R¹, X, and Z are as defined in d.

30. The method of claim 25, wherein R¹, X, and Z are as defined in e.

31. The method of claim 25, wherein R¹, X, and Z are as defined in f.

32. The method of claim 25, wherein the compound is administered via a route selected from the group consisting of oral, parenteral, and intraperitoneal administration.

33. The method of claim 25, wherein the mammal is a human.

34. The method of claim 26, wherein the compound is administered in an effective amount of between about 0.1 μg to about 25 μg mammal per day.

35. The method of claim 34, wherein the compound is administered in an effective amount of between about 0.25 μg to about 1 μg per day.

36. The method of claim 25, wherein the compound is administered topically.
